SEQUENCE LISTING (4 sequences)

SEQ ID NO: 1 - DNA, 21 bp, Artificial Sequence
  Bmp4 forward primer
attcctggtaaccgaatgctg

SEQ ID NO: 2 - DNA, 23 bp, Artificial Sequence
  Bmp4 reverse primer
ccggtctcaggtatcaaactagc

SEQ ID NO: 3 - DNA, 20 bp, Artificial Sequence
  Gapdh forward primer
tgtgtccgtcgtggatctga

SEQ ID NO: 4 - DNA, 21 bp, Artificial Sequence
  Gapdh reverse primer
cctgcttcaccaccttcttga

(19) United States
(12) Patent Application Publication
(10) Pub. No.: US 2020/0049710 A1
(43) Pub. Date: Feb. 13, 2020

(54) ERYTHROPOIETIC ROLE OF RESIDENT MACROPHAGES IN HEMATOPOIETIC ORGANS

(71) Applicant: Albert Einstein College of Medicine, Bronx, NY (US)

(72) Inventors: Andrew Chow, New York, NY (US); Paul S. Frenette, New York, NY (US); Miriam Merad, New York, NY (US)

(73) Assignee: Albert Einstein College of Medicine, Bronx, NY (US)

(21) Appl. No.: 16/508,101

(22) Filed: Jul. 10, 2019

Related U.S. Application Data
(63) Continuation of application No. 14/189,110, filed on Feb. 25, 2014, now abandoned.
(60) Provisional application No. 61/771,391, filed on Mar. 1, 2013.

(51) Int. Cl. G01N 33/569; A61K 35/15; C07K 16/28

(52) U.S. Cl. CPC G01N 33/56972; A61K 35/15; C07K 16/2836; A61K 2039/505; G01N 2800/52; G01N 2800/22; G01N 2333/70596

(57) ABSTRACT

Methods of determining the erythroid prognosis of an anemia, methods of treating a blood disorder in a subject comprising an anemia, and methods of treating a blood disorder in a subject comprising an expanded erythron are all provided.

### CROSS-REFERENCE TO RELATED APPLICATIONS

This application is a continuation application of U.S. patent Ser. No. 14/189,110, filed Feb. 25, 2014, which claims benefit of U.S. Provisional Application No. 61/771,391, filed Mar. 1, 2013, the contents of which are hereby incorporated by reference.

### STATEMENT OF GOVERNMENT SUPPORT

This invention was made with government support under grant numbers F30HL099028, HL097700, HL069438, DK056638, R01HL116340, and RO1CA112100 awarded by the National Institutes of Health. The government has certain rights in the invention.

### BACKGROUND OF THE INVENTION

Throughout this application various publications are referred to in parentheses. Full citations for these references may be found at the end of the specification. The disclosures of these publications, and all patents, patent application publications and books referred to herein, are hereby incorporated by reference in their entirety into the subject application to more fully describe the art to which the subject invention pertains.

Humans produce millions of erythrocytes each minute and careful coordination of production and clearance are critical to maintain erythropoietic homeostasis. This homeostasis can be particularly challenged by a number of genetic (e.g. sickle cell disease, thalassemia, polycythemia vera), infectious (e.g. malaria, parvovirus), exposure (e.g. lead, radiation, trauma-induced blood loss), and iatrogenic (e.g. chemotherapy, bone marrow transplant) perturbations.

In 1958, Marcel Bessis proposed that erythropoietic maturation required a specific microenvironment comprised of a nursing macrophage decorated by erythroblasts at various stages of maturation, culminating with enucleation (1). A functional role for these erythroblastic islands was first demonstrated by Narla and colleagues when they showed that hypertransfused animals had a substantial reduction in the number of islands as quantified by tridimensional electron microscopy (2). A supportive role of macrophages in erythroblast development was strengthened by in vitro observations that macrophages promote erythroblast proliferation and survival (3-5) and an extensive amount of work has been done to characterize the adhesive interactions within these islands (reviewed in 6). Nonetheless, whether macrophages contribute to erythropoiesis in vivo remains to be elucidated.

The present invention addresses the need for improved methods of therapeutic control of erythropoiesis and improved diagnoses for certain blood disorders.

### SUMMARY OF THE INVENTION

This invention provides a method of treating a blood disorder comprising erythropoietic stress in a subject, the method comprising administering to the subject an amount of an erythropoiesis-stimulating agent effective to treat a blood disorder comprising erythropoietic stress.

Also provided is a method of treating a blood disorder comprising an expanded erythron in a subject, the method comprising administering to the subject an amount of a CD169+ macrophage-ablating or CD169+ macrophage-inhibiting agent effective to treat a blood disorder comprising an expanded erythron, or administering to the subject an amount of an BMP4-abrogating agent effective to treat a blood disorder comprising an expanded erythron.

Also provided is a method of determining the prognosis of an erythroid compartment in a subject having erythropoietic stress, comprising obtaining a bone marrow sample and/or splenic sample from the subject and quantifying CD169+ macrophages in the sample(s), comparing the amount of CD169+ macrophages quantified to a predefined reference amount, and determining the prognosis of the erythroid compartment as a negative or a positive prognosis, wherein an amount of CD169+ macrophages quantified in excess of the reference amount indicates a positive prognosis, and an amount of CD169+ macrophages quantified below the reference amount indicates a negative prognosis.

Also provided is a method of preparing a composition comprising obtaining a biological sample comprising CD169+ macrophages, recovering CD169+ macrophages from the sample, and admixing the CD169+ macrophages with a carrier.

Also provided is a composition comprising isolated CD169+ macrophages and a pharmaceutically acceptable carrier.

Also provided is an erythropoiesis-stimulating agent for treating a blood disorder comprising erythropoietic stress.

Also provided is an amount of a CD169+ macrophage-ablating agent or CD169+ macrophage-inhibiting agent for treating a blood disorder comprising an expanded erythron in a subject.

### BRIEF DESCRIPTION OF THE DRAWINGS

FIG. 1A-1H: Depletion of bone marrow CD169+ macrophages results in reduced erythroblast numbers without peripheral blood anemia. a) Photomicrograph of femurs dissected from wild-type (Ctrl) or CD169DTR/+(DTR) treated with DT for 4 weeks. b) Percentage of F4/80+ Ter119+ multiplets (Erythroblast islands) in femurs of Ctrl and DTR mice (n=5). c) Quantitation of BM macrophages at various time points of depletion (n=3-18). Absolute numbers of macrophages per femur were normalized such that average values of Ctrl mice were set at 100% at each time point. d) Flow cytometry plots of DAPI−CD11b−CD45− single cells from BM of Ctrl or DTR mice. e) Quantitation of BM erythroblasts (sum of populations I-IV of d) at various time points of depletion (n=3-18). Absolute numbers of erythroblasts per femur were normalized such that average values of Ctrl mice were set at 100% at each time point. f) Hematocrit measurement from circulating blood after CD169+ macrophage depletion over 6 weeks (n=9-14, pooled from three independent experiments). g) Representative FACS plot and h) quantitation of percentage of DAPI−CD11b−CD45−Ter119+CD71− single cells in peripheral blood that were biotin+ during weekly bleeding of Ctrl and DTR mice (n=5, representative of two independent experiments).

FIG. 2A-2H: Depletion of macrophages impairs erythroid recovery after hemolytic anemia and acute blood loss. a-b) Reticulocyte and hematocrit assessments in Ctrl and DTR mice following induction of hemolytic anemia with phenylhydrazine on days 0 and 1 (n=10, pooled from two independent experiments). c-d) Reticulocyte and hematocrit assessments in PBS and clodronate liposome-treated mice following induction of hemolytic anemia with phenylhydrazine (n=4, representative of two independent experiments). e-f) Reticulocyte and hematocrit assessments in splenectomized Ctrl and DTR mice following induction of hemolytic anemia with phenylhydrazine (n=7-11, pooled from two independent experiments). g-h) Reticulocyte and hematocrit assessments in PBS and clodronate liposome-treated mice following acute bleeding on days 0, 1, and 2 (n=5).

FIG. 3A-3J: Depletion of macrophages impairs erythroid recovery after myeloablation. a-d) Macrophage (a,c) and erythroblast (b,d) counts per femur (a,b) and spleen (c,d) of Ctrl (blue) and DTR (red) animals 7 days after transplantation of 1×10⁶ BM cells. Untransplanted animals (black) are displayed for comparison (BM: n=7-10, pooled from two independent experiments; spleen: n=4-5). e-f) Reticulocyte and hematocrit assessments following transplantation of 1×10⁶ BM cells (n=20, pooled from five independent experiments). g) Gene expression of Bmp4 and h) stress BFU-E in spleens of untransplanted (black), Ctrl (blue), and DTR (red) animals 7 days after BMT (n=3-4). RU=(106)(expression relative to Gapdh). i-j) Quantitation of splenic i) erythroblasts and j) stress BFU-E in reciprocally-transplanted and DT-treated mice 7 days after BMT (n=5).

FIG. 4A-4H: VCAM1 blockade abrogates bone marrow erythroblast recovery. a) FACS plots of surface-bound VCAM1 levels on BM monocytes, BM macrophages and splenic red pulp macrophages (blue=VCAM1, gray=isotype control). b) VCAM1 levels (mean fluorescent intensity, MFI) on BM DAPI-single cells in untransplanted animals (black) or 7 d after BMT in Ctrl (blue) and DTR (red) mice (n=4-5, representative of two independent experiments). c-e) Quantitation of BM c) macrophages per femur, d) VCAM1 MFI and e) erythroblast numbers in reciprocally-transplanted and DT-treated mice 7 d after BMT (n=5). f) BM erythroblast numbers 7 d after BMT of Ctrl (blue), DTR (full red), rat IgG-treated (white) or anti-VCAM1-treated (black) animals (n=3-4). g-h) Reticulocyte and hematocrit assessments in rat IgG-treated (blue) or anti-VCAM1 (red) animals following BMT (n=10).

FIG. 5A-5B: CD15−CD163+CD169+ marks a population of human macrophages expressing VCAM1. a) FACS plots of subpopulations of CD45+ cells from a healthy human BM aspirate sample distinguished by differential expression of CD163, CD15, CD14, CD169, and VCAM1. Representative data from two independent samples are shown. b) Compiled photomicrographs of populations that were sorted as indicated and cytospun. Scale bar=10 μm.

FIG. 6A-6J: Depletion of macrophages normalizes the erythroid compartment in a JAK2V617F-induced murine model of polycythemia vera. a,b) Erythroid parameters from circulating blood counts at 9, 16 and 25 d after transplantation of 3.7×10⁶ wild-type (white, Ctrl) or JAK2V617F (black, PV) bone marrow cells (n=10, pooled from two independent experiments). c-f) Macrophage (c,e) and erythroblast (d,f) counts per femur (c,d) and spleen (e,f) 7 d after last of four weekly infusions of liposomes (day 28 of experiment, 9 weeks post-BMT) into Ctrl or PV animals (n=3). g) Hematocrit levels of Ctrl (black) or PV mice that were treated with PBS (blue) or clodronate (red) liposomes (n=11-13, pooled from two independent experiments). Data analysed with two-way ANOVA with Bonferroni post-test. Day 0 corresponds to first day of liposome injection and 5 weeks after BMT. Liposomes were injected on days 0, 7, 14 and 21 (grey arrows). h-j) Quantitation of h) gene expression of Bmp4, i) stress BFU-E, and j) endogenous BFU-E in spleens of Ctrl (black) and PV mice treated with PBS (blue) or clodronate (red) liposomes (n=4-6) and harvested on day 30 of experiment. RU=(106)(expression relative to Gapdh). Day 0 corresponds to first day of liposome injection and 8 weeks after BMT. Liposomes were injected on days 0, 7, 14 and 21.

### DETAILED DESCRIPTION OF THE INVENTION

This invention provides a method of treating a blood disorder comprising erythropoietic stress in a subject, the method comprising administering to the subject an amount of an erythropoiesis-stimulating agent effective to treat a blood disorder comprising erythropoietic stress.

In an embodiment, the erythropoiesis-stimulating agent comprises an amount of CD169+ macrophages. In an embodiment, the CD169+ macrophages are allogeneic to, or syngeneic to, the subject. In an embodiment, the CD169+ macrophages are obtained from the subject prior to the subject having the erythropoietic stress. In an embodiment, the CD169+ macrophages are isolated. In an embodiment, the CD169+ macrophages are purified. In an embodiment, the erythropoiesis-stimulating agent is a pharmaceutical. In an embodiment, the erythropoiesis-stimulating agent is a small organic molecule of 2000 daltons or less. In an embodiment, the erythropoiesis-stimulating agent is a small organic molecule of 2000 daltons or less which stimulates CD169+ macrophages or stimulates CD169+ macrophage production. In an embodiment, the erythropoiesis-stimulating agent is a human CSF-1 receptor agonist.

In an embodiment, the erythropoietic stress is an anemia. In an embodiment, the erythropoietic stress is, or results from, acute blood loss, acute or chronic hemolysis, a hemoglobinopathy, myeloablative injury, hematopoietic stem cell transplant, chemotherapy or irradiation-induced injury.

Also provided is a method of treating a blood disorder comprising an expanded erythron in a subject, the method comprising administering to the subject an amount of a CD169+ macrophage-ablating or CD169+ macrophage-inhibiting agent effective to treat a blood disorder comprising an expanded erythron, or comprises administering to the subject an amount of an BMP4-abrogating agent effective to treat a blood disorder comprising an expanded erythron. In an embodiment, the blood disorder comprises polycythemia vera.

In an embodiment, the CD169+ macrophage-ablating agent or CD169+ macrophage-inhibiting agent is administered. In an embodiment, the CD169+ macrophage-ablating agent or CD169+ macrophage-inhibiting agent is administered in a manner effective to deliver it to bone marrow of a subject. In an embodiment, the CD169+ macrophage-ablating agent or CD169+ macrophage-inhibiting agent is administered in a manner effective to deliver it to a spleen of a subject. In an embodiment, the CD169+ macrophage-ablating agent or CD169+ macrophage-inhibiting agent is a human CSF-1 receptor inhibitor. In an embodiment, the human CSF-1 receptor inhibitor is an isolated anti-CSF-1 receptor antibody or a human CSF-1 receptor-binding fragment of such an antibody. In an embodiment wherein the human CSF-1 receptor inhibitor is an isolated anti-CSF-1 receptor antibody or a human CSF-1 receptor-binding fragment of such an antibody, the antibodies can be delivered naked, or in a pharmaceutically acceptable carrier, or loaded on dendritic cells. In an embodiment, the human CSF-1 receptor inhibitor is an isolated anti-CSF-1 receptor nucleic acid aptamer. In an embodiment, the CD169+ macrophage-ablating agent is administered. In an embodiment, the CD169+ macrophage-inhibiting agent is administered.

Also provided is a method of determining the prognosis of an erythroid compartment in a subject having erythropoietic stress, comprising obtaining a bone marrow sample and/or splenic sample from the subject and quantifying CD169+ macrophages in the sample(s), comparing the amount of CD169+ macrophages quantified to a predefined reference amount, and determining the prognosis of the erythroid compartment as a negative or a positive prognosis, wherein an amount of CD169+ macrophages quantified in excess of the reference amount indicates a positive prognosis, and an amount of CD169+ macrophages quantified below the reference amount indicates a negative prognosis.

In an embodiment, the CD169+ macrophages are quantified by a flow cytometric method. In an embodiment, the CD169+ macrophages are quantified by a method comprising contacting the macrophages with a labeled anti-human CD169 antibody. In an embodiment, the label is a fluorescent label or a radioactive label. In an embodiment, the method further comprises administering a blood product and/or a erythropoiesis-stimulating agent to a subject identified to be in need thereof by being identified as having negative prognosis by said method. In an embodiment, the blood product is administered as a blood transfusion. In an embodiment, the erythropoiesis-stimulating agent comprises an amount of CD169+ macrophages. In an embodiment, the CD169+ macrophages are allogeneic to, or syngeneic to, the subject. In an embodiment, the CD169+ macrophages are obtained from the subject prior to the subject having the erythropoietic stress. In an embodiment, the erythropoiesis-stimulating agent is a small organic molecule of 2000 daltons or less. In an embodiment, the erythropoiesis-stimulating agent is a small organic molecule of 2000 daltons or less which stimulates CD169+ macrophages or stimulates CD169+ macrophage production. In an embodiment, the erythropoiesis-stimulating agent is a human CSF-1 agonist.

In an embodiment, the erythropoietic stress is an anemia. In an embodiment, the erythropoietic stress results from acute blood loss, acute or chronic hemolysis, a hemoglobinopathy, myeloablative injury, hematopoietic stem cell transplant, chemotherapy or irradiation-induced injury.

In an embodiment of any of the methods described herein, the subject is human.

Also provided is a method of preparing a composition comprising obtaining a biological sample comprising CD169+ macrophages, recovering CD169+ macrophages from the sample, and admixing the CD169+ macrophages with a carrier. In an embodiment, the concentration of CD169+ macrophages in the composition comprising the carrier is greater than the concentration of CD169+ macrophages in the same volume of sample. In an embodiment, the composition is a pharmaceutical composition and the carrier is a pharmaceutically acceptable carrier. In an embodiment, the CD169+ macrophages obtained are optionally cultured with CSF-1 prior to admixing the CD169+ macrophages with a carrier. In an embodiment, the method further comprises concentrating the CD169+ macrophages in a volume prior to admixing with a carrier. In en embodiment, the CD169+ macrophages are enriched in a volume of liquid or carrier relative to their level in an equal volume of a biological sample obtained from a human subject comprising the CD169+ macrophages. In an embodiment, the CD169+ macrophages are expanded prior to admixing with the carrier. In an embodiment, the CD169+ macrophages are cultured with CSF-1 prior to admixing with the carrier. In an embodiment, the CSF-1 is recombinant human CSF-1.

Also provided is a method of preparing a composition comprising obtaining a biological sample of bone marrow from a subject, culturing said bone marrow with CSF-1, subsequently identifying and recovering CD169+ macrophages from the sample, and admixing the CD169+ macrophages with a carrier.

In an embodiment of the methods of preparing, the CD169+ macrophages are obtained from a biological sample obtained from a human subject.

Also provided is a composition comprising isolated CD169+ macrophages and a pharmaceutically acceptable carrier. In an embodiment, the isolated CD169+ macrophages are enriched in the composition relative to the same volume in a human bone marrow or human splenic sample.

Also provided is an erythropoiesis-stimulating agent for treating a blood disorder comprising erythropoietic stress. In an embodiment, the erythropoiesis-stimulating agent comprises an amount of CD169+ macrophages or is a CSF-1 agonist. In an embodiment, the erythropoiesis-stimulating agent comprises an amount of CD169+ macrophages and the CD169+ macrophages are allogeneic to, or syngeneic to, the subject. In an embodiment, the erythropoietic stress is an anemia. In an embodiment, the erythropoietic stress is, or results from, acute blood loss, acute or chronic hemolysis, a hemoglobinopathy, myeloablative injury, hematopoietic stem cell transplant, chemotherapy or irradiation-induced injury.

Also provided is an amount of a CD169+ macrophage-ablating agent or CD169+ macrophage-inhibiting agent for treating a blood disorder comprising an expanded erythron in a subject.

In an embodiment of the CD169+ macrophage-ablating agent or CD169+ macrophage-inhibiting agent of, the blood disorder comprises polycythemia vera. In an embodiment of the CD169+ macrophage-ablating agent or CD169+ macrophage-inhibiting agent, the CD169+ macrophage-ablating agent or CD169+ macrophage-inhibiting agent is formulated for administration to bone marrow and/or spleen of a subject. In an embodiment of the CD169+ macrophage-ablating agent or CD169+ macrophage-inhibiting agent, the CD169+ macrophage-ablating or CD169+ macrophage-inhibiting agent is a human CSF-1 receptor inhibitor. In an embodiment of the CD169+ macrophage-ablating agent or CD169+ macrophage-inhibiting agent, the human CSF-1 receptor inhibitor is an isolated anti-CSF-1 receptor antibody or a human CSF-1 receptor-binding fragment of such an antibody, or is a human CSF-1 receptor-binding nucleic acid aptamer.

As used herein, an “erythroid compartment” is a portion of, or the whole of, the cells in a subject that are red blood cells or precursors thereof.

As used herein, “erythropoietic stress” is a state in which erythropoiesis in a subject is sub-optimal and/or is insufficient for the erythropoietic health of a subject. Such may occur due to a variety of causes, as known in the art, including, but not limited to, anemia, acute blood loss, and myeloablative injury.

As used herein, a “predefined reference amount” is a control amount. The concept of a control is well-established in the field, and can be determined, in a non-limiting example, empirically from non-afflicted subjects (versus afflicted subjects). The control amount may be normalized as desired to negate the effect of one or more variables, including sample size.

In an embodiment the macrophage-ablating agent or macrophage-inhibiting agent comprises clodronate or a pharmaceutically acceptable clodronate salt.

In an embodiment, “determining” as used herein means experimentally determining.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

It was recently reported that murine BM macrophages express CD169 (also known as Sialoadhesin or Siglec-1) (7, 8) and that these macrophages can be selectively depleted in CD169-DTR mice, which express the human diphtheria toxin receptor (DTR) knocked-in downstream of the endogenous Siglecl promoter (9). Since central macrophages in erythroblastic islands reportedly express CD16910, it was sought to re-examine the role of macrophages in steady-state erythropoiesis in vivo. Moreover, the contribution of BM and splenic macrophages to the recovery from erythropoietic stress and also to the hyperfunctional erythron observed in JAK2V617-induced polycythemia vera was assessed.

### Results

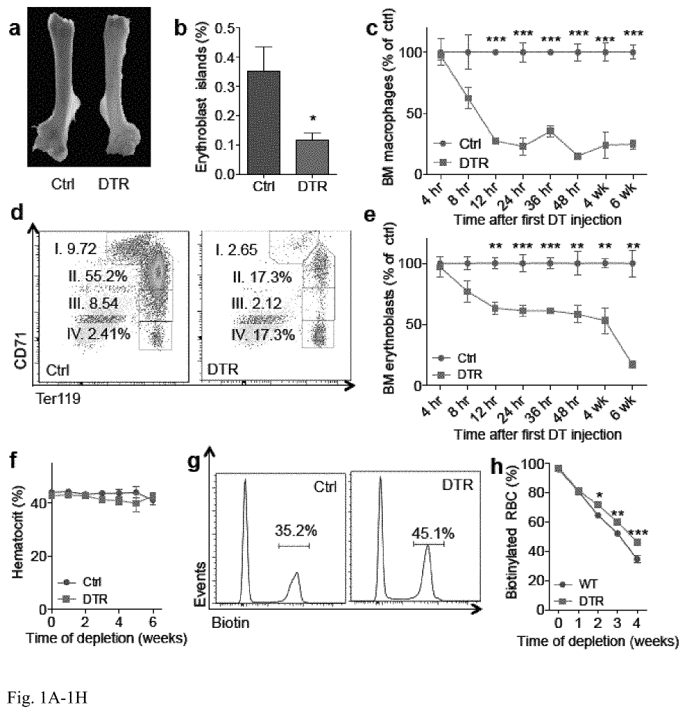

CD169+ macrophage depletion reduces bone marrow erythroblasts, but does not result in peripheral blood anemia: To examine the role of BM macrophages in erythroblast formation, heterozygous CD169-DTR (CD169DTR/+) animals were utilized. It was observed that after sustained diphtheria toxin (DT) administration and ensuing depletion of BM CD169+ macrophages, but not monocytes (7,8), long bones were paler than that of control animals (FIG. 1a), and this was associated with a >60% reduction in the number of F4/80+Ter119+ erythroblast islands (FIG. 1b). It was also observed that sustained depletion of CD169+ macrophages (FIG. 1c) resulted in a reduction in erythroblasts in the BM at various time points after initiation of depletion, starting as early as 12 h post DT administration (with two gating schemes: FIG. 1c-e). This reduction in BM erythroblasts was observed across all stages of maturation. Cultured erythroblasts from CD169DTR/+ mice were unaffected by DT administration, whereas cultured macrophages were susceptible at the same dose (7 and data not shown), ruling out a direct depletion of erythroblasts. Consistent with the flow cytometry data, a ˜50% reduction was observed in CFU-E, but not BFU-E, in the BM 24 h after DT administration, which is consistent with the notion that macrophages are important for the BM erythroblast stages starting with the CFU-E/proerythroblasts (6,11). The reduction of erythroblasts was not due to lower proliferation or viability. The observation that CD169+ macrophages control the retention of hematopoietic stem and progenitor cells in the BM7 together with the finding that the reduction of BM CD169+ macrophages and erythroblasts follow similar kinetics in the CD169DTR/+ depletion model (FIG. 1c,e) led the inventors to hypothesize that increased release of erythroblasts into the peripheral circulation might account for the erythroblast reduction in the BM. Indeed, 24 h after initiation of CD169+ macrophage depletion, >2-fold increase in the number of erythroblasts in the peripheral blood (PB) was observed, which is consistent with a prior report (12). Although PB erythroblasts were similarly viable, they proliferated half as much as their BM counterparts. This increased level of circulating peripheral EB is sustained after four weeks of depletion and all four subsets of EB are increased. Since the spike in PB erythroblasts alone cannot account for the reduction observed in the BM, the erythroblasts are presumably mobilized to the spleen and other unexamined peripheral tissues. Thus, although BM CD169+ macrophages do not regulate erythroblast proliferation or viability per se in the steady-state BM, they control their retention in the BM, which represents a major site for erythropoiesis.

Despite a reduction of BM erythroblasts, mice did not develop an overt peripheral blood anemia (FIG. 1f), consistent with previous reports utilizing clodronate liposomes (12,13). CD169+ macrophage-depleted animals did not have a compensatory increase in serum erythropoietin or hepatic erythroblastosis. Although compensatory splenic erythroblastosis was present, splenectomized CD169+ macrophage-depleted mice also did not develop overt anemia, indicating that splenic compensation was not sufficient to mask the BM production defect. Along with BM macrophages, splenic red pulp macrophages (RPM) and hepatic Kupffer macrophages were also reduced after four weeks of sustained DT administration. Consistent with the fact that the latter two populations are critical for the clearance of aged red blood cells (RBCs) (14), it was observed that CD169+ macrophage depletion resulted in a ˜25% increase in RBC lifespan after 4 weeks, suggesting that abrogated clearance of aged RBCs was a mechanism parallel to splenic compensation that masked the reduction of BM erythroblasts in the steady state. Mathematical modeling was used to assess whether the prolongation of RBC lifespan was sufficient to explain the absence of anemia after macrophage depletion. Since macrophages were involved in both the production and clearance of RBCs, the analysis suggested that peripheral RBC counts in the steady state are proportional to the ratio between the rates of production and clearance and independent of the absolute macrophage content.

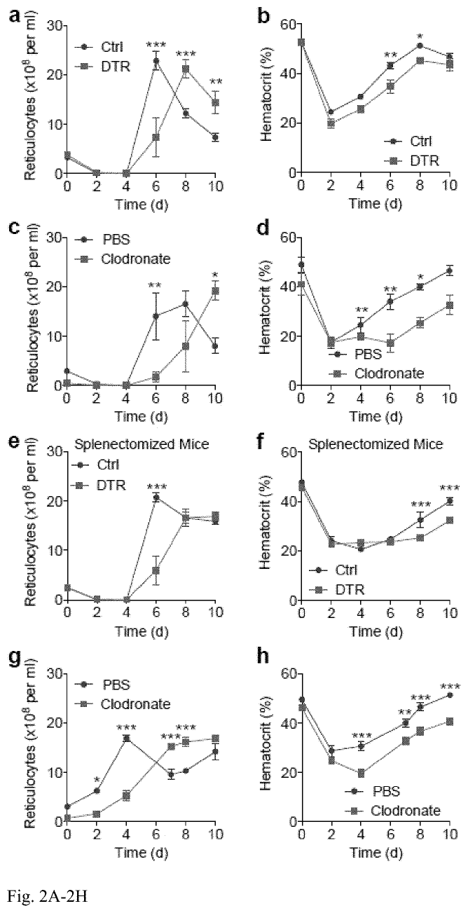

Bone marrow and splenic macrophages are critical for recovery from hemolytic anemia and acute blood loss: Although no anemia developed from CD169+ macrophage depletion in steady-state animals, it was reasoned that a difference might be resolved after erythropoietic stress. Indeed, in a model of hemolytic anemia induced by the hemoglobin-oxidizing toxin phenylhydrazine (PHZ), a delay was observed in reticulocytosis and hematocrit recovery in macrophage-depleted animals using both the CD169-DTR and clodronate liposome model which depletes most mononuclear phagocytes, including BM monocytes and CD169+ macrophages (7) (FIG. 2a-d). Since clodronate liposomes appeared to more dramatically impair the recovery from hemolytic anemia compared to the CD169-DTR model, it was reasoned that this could be due to differential capacity of the two models to deplete splenic RPM. Indeed, whereas both models efficiently depleted BM macrophages (7), short-term administration of DT did not result in a reduction in splenic RPM. This is consistent with a prior report (9) and in contrast with the reduction observed after four weeks of DT administration.

Pre-treatment of clodronate liposomes five days prior to the administration of PHZ reduced macrophage numbers and impaired recovery of erythroblasts in the BM and spleen. Moreover, macrophage depletion reduced splenic BMP4 induction and the number of splenic stress BFU-E. The impairment in hematocrit recovery from PHZ challenge, albeit more modest in the CD169-DTR model, suggested that BM erythropoiesis made a functional contribution to recovery from hemolytic anemia, along with their splenic stress counterparts. To further ascertain the contribution of BM erythropoiesis, recovery of control and CD169+ macrophage-depleted splenectomized mice was compared. It was observed that although hematopoietic recovery was slower compared to non-splenectomized animals (FIG. 2b,f), CD169+ macrophage-depleted animals still demonstrated a hampered recovery (FIG. 2e,f). Consistent with the PHZ model and a prior report (15), macrophage-depleted animals also demonstrated a substantial impairment in recovery from acute blood loss (FIG. 2g,h). Hence, in two models of acute RBC reduction, macrophages are essential for efficient recovery.

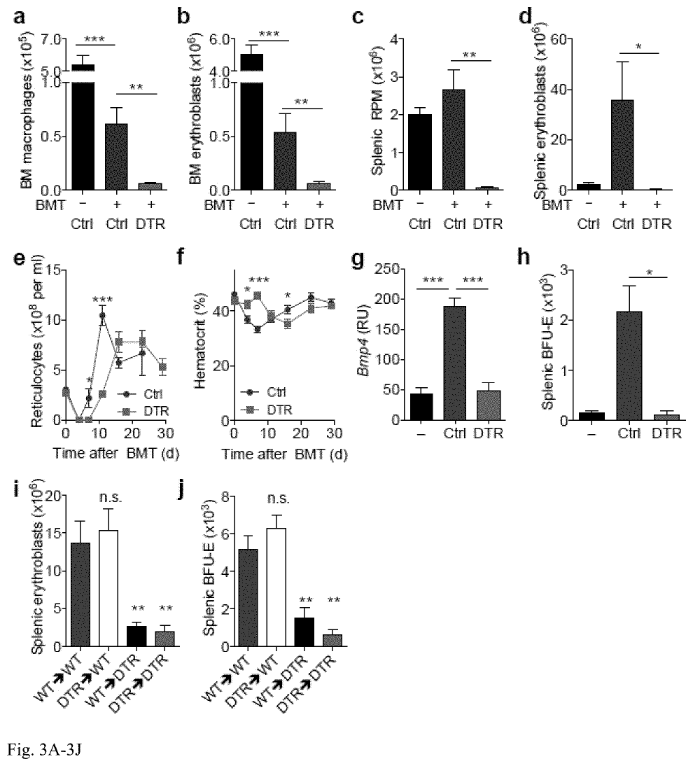

Radioresistant splenic red pulp macrophages are critical for BMP4-dependent stress erythropoiesis and erythroid recovery following myeloablation: To test whether CD169+ macrophages could also contribute to erythroid recovery from myeloablation, mice were depleted after bone marrow transplantation (BMT). BMT itself reduced the number of BM CD169+ macrophages and erythroblasts seven days after BMT (FIG. 3a,b), and the reduction in erythroblasts was even more profound when CD169+ macrophages were depleted following BMT (FIG. 3b). Moreover, CD169+ macrophage depletion post-BMT severely abrogated the recovery of splenic erythroblasts (FIG. 3c,d). Thus, in the context of myeloablation, splenic RPM are efficiently depleted by short-term DT administration in the CD169-DTR model. CD169+ macrophage depletion also delayed reticulocytosis and hematocrit recovery (FIG. 3e,f), indicating the functional peripheral consequences of impaired erythroblast recovery. CD169+ macrophage depletion was similarly associated with delayed erythroblast and peripheral erythrocyte recovery following challenge with the myeloablative agent 5-fluorouracil (5FU). Interestingly, in both BMT and 5FU models, CD169+ macrophage-depleted animals had less severe early declines in hematocrit (FIG. 3f. This suggests that myeloablation-induced pathogenic consumption of mature RBC by macrophages may contribute to anemia following clastogenic injury, which was confirmed by observing that RBCs had a longer half-life in CD169+ macrophage-depleted animals shortly after BMT. Together, these results indicate that similar to the steady state, CD169+ macrophages promote both the production and destruction of erythrocytes. Nonetheless, the supportive role of CD169+ macrophages in erythroid production is both dominant and essential for efficient recovery from myeloablation.

It was initially hypothesized that macrophages may represent nurse-like cells providing iron to developing RBC16 and since then, macrophage regulation of iron homeostasis has been well-documented (17). Serum iron, transferrin saturation, mean corpuscular hemoglobin (MCH), and reticulocyte hemoglobin content (CHr) were analyzed in the steady state or following BMT to evaluate the potential effect of CD169+ macrophage depletion on iron homeostasis in erythrocytes. In the steady state, CD169+ macrophage depletion reduced serum iron, transferrin saturation, MCH and CHr after 3-4 weeks of sustained depletion. This delayed effect is more consistent with compromised ferroportin-mediated systemic iron recycling by macrophages (18), rather than a local nurse-like function. In the context of erythropoietic challenge from BMT, no significant changes were observed in serum iron or transferrin saturation 7 d after transplant and MCH did not show a reduction until the third week post-BMT; however, a reduction in CHr could be observed by seven days following BMT, suggesting a local role of tissue macrophages in iron homeostasis in the early recovery from myeloablation, which is consistent with a local nurse-like role. Systemic administration of iron dextran did not rescue the impaired erythropoietic recovery observed in CD169+ macrophage-depleted animals. Although these data do not preclude a local role of macrophage-derived iron in the observed deficits, they do suggest that macrophages alter the erythron through additional mechanisms.

Since BMP4 promotes the development of stress erythroid progenitors following BMT (19,20), whether induction of splenic BMP4 was reduced in CD169+ macrophage-depleted animals was assessed. Indeed, it was found that splenic induction of BMP4 and stress BFU-E was abrogated in CD169+ macrophage-depleted animals (FIG. 3g,h). Splenic RPM are radioresistant compared to other hematopoietic populations (21,22) and have been previously implicated as the source of BMP423. Since 90% of splenic RPM remained of host origin seven days after BMT, whether depletion of host-derived splenic RPM was sufficient to abrogate erythropoietic recovery was assessed by performing reciprocal BMT between WT and CD169DTR/+ animals and treating all mice with DT. CD169DTR/+ animals transplanted with WT BM cells (WT→DTR) demonstrated similar levels of depletion of splenic RPM compared to those transplanted with CD169DTR/+BM cells (DTR→DTR), confirming the predominance of host-derived RPM seven days after BMT. Importantly, WT→DTR animals also had impaired recovery of splenic erythroblasts and stress BFU-E (FIG. 3i,j). Taken together, BMP4 derived from radioresistant, host-derived splenic RPM is critical for erythroid recovery following myeloablation.

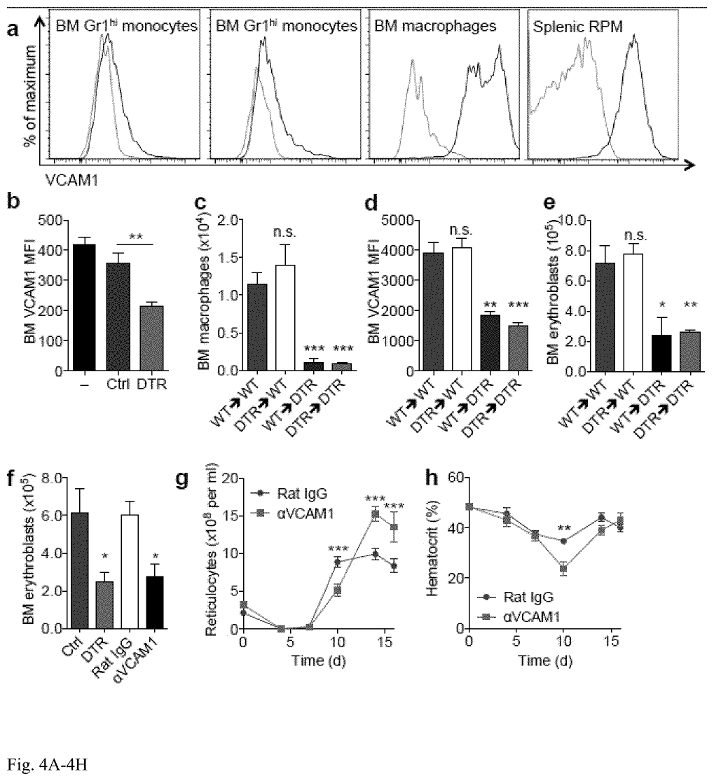

Abrogation of VCAM1 impairs erythropoietic recovery following myeloablation: Vascular cell adhesion molecule 1 (VCAM1) has previously been demonstrated to play a role in erythroblast island interactions in vitro (24). Gene expression profiling of purified BM mononuclear phagocytes revealed that the expression of Vcam1 transcripts was significantly higher on BM CD169+ macrophages compared to BM Grlhi or Grllo monocytes. Consistently, monocytes expressed low VCAM1 levels on the cell surface, whereas both BM and splenic RPM25 expressed abundant levels of VCAM1 (FIG. 4a). In addition, cell-surface levels of VCAM1 were reduced in the BM of CD169+ macrophage-depleted mice in the steady state and seven days post-BMT (FIG. 4b). In line with the role of radioresistant host-derived macrophages in the spleen, it also observed that depletion of radioresistant host-derived BM CD169+ macrophages in the reciprocal BMT model was sufficient to reduce CD169+ macrophages, VCAM1 levels, and erythroblasts in the BM (FIG. 4c-e). Importantly, anti-VCAM1 antibody administered in the post-BMT setting in macrophage-sufficient animals led to impaired recovery of BM erythroblasts, reticulocytes, and hematocrit, similar to macrophage-depleted animals (FIG. 4f-h). Notably, splenic VCAM1 levels were not dramatically reduced by CD169+ macrophage depletion and anti-VCAM1 antibody did not abrogate the development of splenic erythropoiesis. These data suggests that VCAM1 expressed by CD169+BM macrophages works in parallel with BMP4 derived from CD169+ splenic macrophages to promote erythroid recovery following myeloablation.

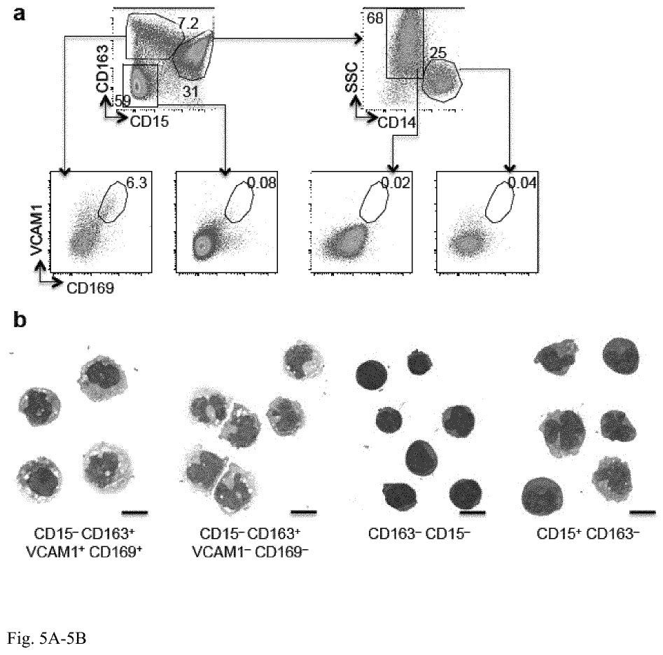

Human BM macrophages co-express CD169 and VCAM1: To determine whether human BM macrophages shared features with their murine counterparts, phenotypic analysis of cells from the BM aspirate of healthy donors was performed and assessed for CD169 and VCAM1 expression. CD15 is a marker of human granulocytes and monocytes (26) (FIG. 5a,b), and neither CD15+CD14− granulocytes nor CD15+CD14+ monocytes expressed CD169 or VCAM1. CD163 is a marker of human monocytes and macrophages (27). Within the CD15−CD163+ population, a CD169+ VCAM1+ population with macrophage morphology was present (FIG. 5a,b), whereas the CD169− VCAM1− population appeared to have a monocytic morphology. Therefore, like their murine counterparts, human BM macrophages can also be identified by CD169 and VCAM1 expression.

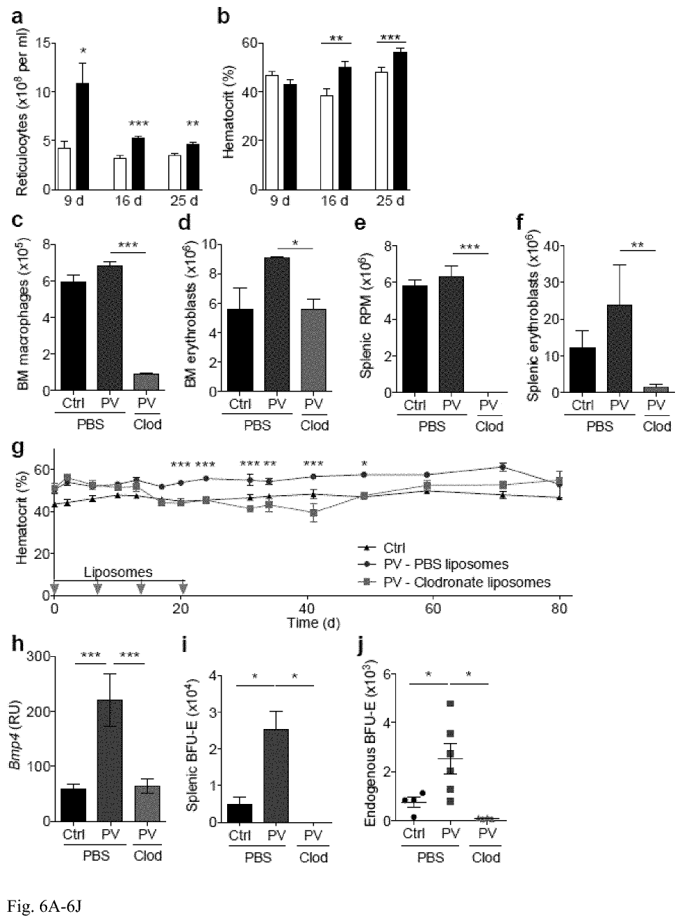

Macrophage depletion normalizes the erythron in JAK2V617F-mediated polycythemia vera: Having demonstrated the role of macrophages in recovery after erythropoietic insufficiency, it was sought to determine whether macrophage depletion could be beneficial in the context of an overactive erythron and tested the effect of depletion in a model of polycythemia vera (PV). It was hypothesized that even when driven by an oncogenic mutation, erythropoiesis might still respond to microenvironmental cues from its niche. To investigate this issue, BM cells isolated from wild-type (WT) mice or transgenic mice harboring the JAK2V617F mutation (28) were transplanted into lethally irradiated wild-type mice. Increased reticulocytosis was already observed by day 9 post-BMT (FIG. 6a) and erythrocytosis was observed by day 16 post-BMT (FIG. 6b), whereas WBC and platelet recovery were not consistently different. Five weeks after BMT, recipients of JAK2V617F BM (PV mice) were infused weekly with PBS- or clodronate-encapsulated liposomes for 4 weeks. Macrophage depletion reduced erythroblasts in the BM and spleen (FIG. 6c-f), affecting all splenic erythroblast subsets, and strikingly normalized blood hematocrit (FIG. 6g). The therapeutic benefit of macrophage depletion persisted for four weeks after the cessation of liposome treatment, and a single administration of clodronate liposomes was sufficient to reduce macrophages and erythroblasts in the BM and spleen and normalized the hematocrit for a shorter period than weekly administration.

Macrophage depletion had a subtle effect on MCH, serum iron, and transferrin saturation in the PV model, but a rapid effect on CHr. Although treatment of PV mice with the iron chelating agent deferoxamine reduced serum iron levels, it neither reduced splenic erythroblast numbers nor hematocrit. Hence global alterations in iron are not the mechanism by which macrophage depletion suppresses PV, although this does not necessarily rule out a local microenvironmental effect.

Since splenic erythropoiesis was reduced by macrophage depletion, it was hypothesized that JAK2V617F mutation could potentially induce splenic stress erythropoiesis. Consistent with this hypothesis, BMP4 and stress BFU-E induction was observed in PV animals (FIG. 6h,i), and importantly, clodronate treatment abrogated this induction (FIG. 6h,i). Strikingly, it was also observed that the number of EPO-independent endogenous erythroid colonies, a clinical criterion of JAK2V617F-induced PV29, was markedly reduced after macrophage depletion (FIG. 6j). All together, it is demonstrated for the first time that targeting of macrophages is a novel therapeutic strategy for management of polycythemia vera, a disease commonly thought to be cell-autonomous.

### DISCUSSION

Although erythroblastic islands were the first described hematopoietic niche, the in vivo relevance of this microenvironment for developing RBCs has been unclear. In this study, the dual roles that tissue resident macrophages have in RBC production and clearance are identified. Although these antagonistic roles offset in the steady state, it is demonstrate that the supportive role of macrophages in RBC development is dominant in recovery from hemolytic anemia, acute blood loss, myeloablation, and also JAK2V617F-induced polycythemia vera.

The delay in erythroid progenitor recovery from hemolytic anemia observed in macrophage-depleted animals is consistent with the impairment previously reported in Mx1-Cre; Itga4fl/fl, Mx1-Cre; Itgb1fl/fl, and Tie2-Cre;Vcam1fl/fl animals (30-32). Together, this indicates that binding of erythroblast integrins to VCAM1 on the central macrophage surface promotes recovery from hemolytic anemia. However, despite the delay in erythroid progenitor recovery, defects in erythroid integrins (31,32) do not impact peripheral erythrocyte recovery from hemolytic anemia to the same extent as macrophage depletion, suggesting additional adhesion-independent mechanisms.

In the myeloablative setting, it was observed that depletion of radioresistant host-derived CD169+ macrophages impaired recovery of BM and splenic erythroblasts, which is in line with the tight correlation between the recovery rates of macrophages and erythroid progenitors following allogeneic BMT in humans (33,34). Antibody blockade of VCAM1 was able to reproduce the delayed BM erythroblast recovery observed in CD169+ macrophage-depleted animals, implicating the structural importance of VCAM1 on the surface of CD169+ macrophages in promoting erythroblast recovery after BMT. Human CD169 has a predicted 72% sequence homology to its murine counterpart, and it can be found on human BM resident, splenic red pulp, and liver macrophages (35). Here, it is reported that CD169 and VCAM1 co-expression can also be found on a population of CD15−CD163+ cells in human BM aspirates with macrophage morphology, indicating a similarly phenotyped population in humans.

It has been reported that stress erythropoiesis in mice is dependent on BMP4, which works in concert with stem cell factor, EPO, and hypoxia signals (20). Flex-tailed mice, which have a mutation in the BMP4 downstream target Smad5, have impaired development of stress erythroid progenitors (36) and display severe impairment in peripheral erythroid recovery from hemolytic anemia (37). It was observed that clodronate liposome pre-treatment impairs BMP4 induction, delays development of stress BFU-E, and severely compromises peripheral erythroid recovery from hemolytic anemia, which is consistent with the requirement of macrophages to mount BMP4-mediated stress erythropoiesis. It was also observed that depletion of CD169+ macrophages following BMT could abrogate the development of BMP4-dependent stress erythropoiesis in the spleen. Since CD169+ macrophage-depleted animals phenocopy the erythroid-specific impairment in recovery post-BMT reported in flex-tailed mice (19,20), this suggests that BMP4 derived from splenic RPM (23) promotes stress erythropoiesis in the spleen. Taken together, this supports a model in which VCAM1 expressed on host-derived BM CD169+ macrophages and BMP4 derived from host-derived splenic RPM work in concert to mediate erythrocyte recovery following myeloablation. Persistent anemia following clinical hematopoietic stem cell transplant is a serious concern with currently no optimal solutions (38,39). Blood transfusions are associated with iron overload and increased risk of infections, while erythropoietin supplementation does not reduce the number of transfusions required (40). Thus, strategies to boost CD169+ macrophage recovery following chemotherapy or irradiation-induced injury represents a novel approach to accelerate recovery of the RBC compartment after transplant.

In contrast to myeloablated individuals, patients with PV have a hyperfunctional erythron, resulting in increased blood viscosity and a substantial incidence of thrombosis (41). The current standard of care treatment for PV patients is still phlebotomy (41). JAK2 inhibitors to suppress PV are under clinical trials, but are limited at the moment by dose-dependent toxicity and evidence that resistance can develop (42). In the PV model, it was observed unexpectedly that macrophage depletion could normalize the expanded erythron. This is the first report of BMP4 and stress erythropoiesis contributing to the pathogenesis of PV in mice, and also the first time it has been shown that macrophage depletion abrogates this erythroid expansion. Importantly, it is shown that EPO-autonomous colonies, a diagnostic criterion of PV29, were reduced with macrophage depletion. Thus, the data indicate that inhibition of the macrophage compartment (e.g. CSF-1 inhibitors (43)) or abrogation of BMP4 are new therapies for polycythemia vera.

The dual roles of macrophages in steady-state erythropoiesis are demonstrated herein, and their importance in hemolytic anemia, acute blood loss, myeloablative injury, and polycythemia vera shown.

### Materials and Methods

Mice. All experiments were performed on 8-12 week old animals. C57BL/6 (CD45.2) mice were bred in-house or purchased from Charles River Laboratories (Frederick Cancer Research Center, Frederick, Md.). For JAK2V617F experiments, C57BL/6-Ly5.2 (CD45.1) animals were purchased from Charles River Laboratories. CD169-DTR9 heterozygous (CD169DTR/+) mice, which were generated with DTR cDNA44, were bred in-house by crossing CD169DTR/DTR with C57BL/6 mice. With the exception of the JAK2V617F animals, which were housed at the University of Oklahoma Health Sciences Center, all mice were housed in specific pathogen-free facilities at the Mount Sinai School of Medicine or Albert Einstein College of Medicine animal facility. Experimental procedures performed on the mice at each site were approved by the respective Institutional Animal Care and Use Committee of the Mount Sinai School of Medicine or Albert Einstein College of Medicine.

Macrophage depletion. For depletion of CD169+ macrophages, heterozygous CD169-DTR (CD169DTR/+) were injected i.p. with 10 μg/kg DT (Sigma). For steady-state experiments, mice were injected with a single dose of DT or twice weekly for sustained depletion. For PHZ experiments, animals were injected with DT on days −2, 0, 2, 4, and 6 of experiment (PHZ on days 0 and 1). For BMT and 5FU experiments, DT was administered every three days starting one day after BMT or 5FU administration. C57BL/6 mice injected with DT and CD169DTR/+ mice not injected with DT both did not demonstrate macrophage depletion and were pooled as control (Ctrl) animals. CD169DTR/+ animals injected with DT served as macrophage-depleted experimental mice (DTR). Analysis of macrophage depletion in the CD169DTR/+ model beyond six weeks is not possible due to development of immunity to diphtheria toxin (data not shown). In some experiments, macrophages were depleted by injection of PBS- or clodronate-encapsulated liposomes (200 μl i.v./infusion). C12MDP (or clodronate) was a gift from Roche Diagnostics (GmbH, Mannheim, Germany). For phenylhydrazine and acute bleeding experiments, a single infusion of liposomes was administered on day −5 of experiment. For PV experiments, a single (day 0 of experiment) or four doses (days 0, 7, 14, 21) were administered as indicated in the text.

CBC analysis. Animals were bled ˜25 μl via submandibular route into an eppendorf tube containing 1 μl of 0.5M EDTA (Fisher). Blood was diluted 1:10 in PBS and ran on Advia counter (Siemens).

Cell preparation. Nucleated single cell suspensions were enriched from peripheral blood, bone marrow, spleen and liver by harvesting interface layer from a lympholyte gradient (Cedar Lane Labs), according to manufacturer's directions. For peripheral blood, 250-500 μl of peripheral blood was diluted in 2 ml of RPMI media (Cellgro) and carefully pipetted onto 3 ml of lympholyte solution in a 15 ml tube (Falcon). For BM, femurs were flushed gently with 500 μl of ice-cold PBS (Cellgro) through a 1 ml syringe (BD) with 21G needle (BD) into an eppendorf tube; then, the entire solution was carefully layered onto 1 ml of lympholyte solution in a 5 ml polystyrene tube (BD). Spleens were mashed through a 40 μm filter (BD) onto a 6 well-plate (BD) containing 4 ml of ice-cold PBS. Cell suspension was resuspended to approximately 20×10⁶ cells/ml and 500 μl was layered onto 1 ml of lympholyte solution in a 5 ml polystyrene tube (BD). Liver cells were mechanically diced and digested in a RPMI media (Cellgro) solution containing 0.4 mg/ml Type IV collagenase (Sigma) and 10% FBS (Stem Cell Technologies) for 1 hr. The liver suspension was drawn through a 3 ml syringe (BD) with 19G needle (BD) and filtered through a 40 μm filter (BD). The cells were resuspended in 1 ml PBS and centrifuged on a 30% Percoll gradient. The supernatant was discarded and the pellet was resuspended in 500 μl and was layered onto 1 ml of lympholyte solution in a 5 ml polystyrene tube (BD). For FACS analyses, RBC lysis with ammonium chloride was not used since some erythroblasts became DAPI+ after lysis.

In vivo isolation of erythroblast islands. Protocol was modified from 3. Bone marrow was flushed gently with IMDM media (Cellgro) containing 3.5% sodium citrate and 20% FCS solution using an 18G syringe (BD). After pipetting 20 times, 8% of BM by volume (˜1×10⁶ cells) was incubated with F4/80-FITC and Ter119-PE antibody at 1:100 for two hours at room temperature. Cells were then diluted 3.5-fold in FACS buffer containing DAPI and processed by flow cytometry or flow-sorted for the F4/80+Ter119+ multiplet population by BD FACSAria. Images of erythroblast islands were acquired from glass slides containing 10,000 islands cytospun at 500 rpm for 3 min with a Cytospin 4 (Thermo Scientific).

Flow cytometry. Fluorochrome-conjugated or biotinylated mAbs specific to mouse Gr-1 (Ly6C/G) (clone RB6-8C5), CD115 (clone AFS98), B220 (clone RA3-6B2), VCAM1 (clone 429), CD11b (clone M1/70), CD45 (clone 30-F11), CD45.1 (clone A20), CD45.2 (clone 104), Ter119 (clone TER-119), CD71 (clone R17217), and CD44 (clone IM7), corresponding isotype controls, and secondary reagents (PerCP-efluor710 and PE-Cy7-conjugated Streptavidin) were purchased from Ebioscience. Anti-F4/80 (clone CI:A3.1) was purchased from AbD Serotec. BrdU incorporation of erythroblasts was assessed in animals injected with 100 μg of BrdU i.p. 1 hour prior to harvest and samples were processed according to manufacturer's directions in the APC BrdU Kit (BD Biosciences). In some experiments, APC-conjugated anti-BrdU (clone Bu20a) from Biolegend was used. Positive staining was gated in reference to cells from mice that were not injected with BrdU. Viable cells were assessed by double negative staining of DAPI (1 mg/ml solution diluted to 1:20,000) and Annexin V (BD Biosciences). Samples were processed according to manufacturer's directions, but DAPI was substituted for propidium iodide. In some experiments, Alexa Fluor 647 Annexin V was used according to manufacturer's instructions (Biolegend). For nuclear staining in non-permeabilized cells, cell suspensions were incubated 1:1000 with 10 mg/ml Hoechst 33342 solution (Sigma) for 45 minutes at 37° C. after cell surface staining with other antibodies. For human BM characterization, the following anti-human antibodies were used: VCAM1-PE (clone STA, Biolegend), CD169-Alexa 647 (clone?-239, Biolegend), CD163-biotin (clone eBioGHI/61, Ebioscience), CD15-PerCP-eFluor710 (clone MMA, Ebioscience), and CD14-eFluor450 (clone 61D3, Ebioscience, Biolegend). Multiparameter analyses of stained cell suspensions were performed on an LSRII (BD) and analyzed with FlowJo software (Tree Star). DAPI-single cells were evaluated for all analyses except for peripheral blood erythroblasts and BrdU assessments.

In vitro culture of erythroblasts. DAPI-CD1 lb−CD45−Ter119+CD71+ erythroblasts from wild-type or CD169DTR/+ mice were sorted by FACS Aria (BD) and cultured for 24 or 48 hours, as previously described (45) at a concentration of 1×10⁵ sorted cells per 100 μl in a 96 well plate (BD). Some wells were incubated with 1 μg/ml DT. At 24 and 48 hours after culture, cells were counted and assessed for viability by Annexin-DAPI-staining.

Splenectomy. Animals were splenectomized as previously described (46) and allowed to recover at least two weeks prior to the onset of experiments.

Serum erythropoietin. Serum was frozen and assessed by serum EPO ELISA kit (R&D) according to manufacturer's directions.

In vivo biotinylation assay. Mice were injected i.v. with 100 mg/kg NHS sulfo-biotin (Thermo Scientific—Pierce) on day 0 and lifespan of RBCs was assessed weekly (47) by staining 1 μl of peripheral blood with Streptavidin-PE-Cy7 and gating CD11b-CD45-Ter119+CD71− cells. For BMT mice, mice were infused with NHS sulfo-biotin 1 day prior to BMT.

Erythroid colony-forming assays. BFU-E (Stem Cell Technologies, M3436) and CFU-E (M3334) of BM cells were plated according to manufacturer's instructions and counted on days 10 and 3 of culture, respectively. Splenic stress BFU-E were assayed by plating 0.5×10⁶ RBC-separated splenocytes in M3436 media and enumerating after 5 days of culture. Endogenous (i.e. without EPO) BFU-E and CFU-E were assayed by plating 0.5×10⁶ RBC-separated splenocytes in M3234 media and enumerating after 5 days of culture.

Phenylhydrazine-induced hemolytic anemia. Mice were infused with 40 mg/kg phenylhydrazine for two consecutive days, which were considered days 0 and 1 of the experiment. For BMP4 imaging in PHZ-challenged animals, mice were administered a single dose of 40 mg/kg of PHZ on day 0 and harvested 24 hours later.

BMP4 immunofluorescence. Spleens were harvested, cut into two halves along the longitudinal axis, fixed for 2 h in 4% PFA, then frozen in OCT compound (Sakura), which were subsequently stored at −80° C. 8 μm sections were cut onto Superfrost Plus slides and stained with 1:100 F4/80-biotin (clone CI:A3-1, Serotec) and 1:100 polyclonal rabbit anti-mouse BMP4 (Abcam) for 2 h. Endogenous biotin was blocked with the Avidin/Biotin blocking kit (Vector Laboratories). After washing for 30 minutes with PBS, slides were stained for 1 h with 1:200 Cy5-conjugated Streptavidin (Jackson Labs) and 1:200 Alexa 594-conjugated goat anti-rabbit antibody (Molecular Probes). After washing for 30 minutes with PBS, slides were stained with 2 μg/ml DAPI solution for 10 minutes. Images were acquired on a Zeiss Axioplan 21E equipped with a camera (AxioCam MR).

Acute blood loss. Mice were bled 400 μl under isoflurane anesthesia and immediately volume-repleted intraperitoneally with 500 μl of PBS on days 0, 1, and 2 of experiment.

Bone marrow transplantation. Mice were irradiated (1,200 cGy, two split doses, 3 h apart) in a Cesium Mark 1 irradiator (J L Shepperd & associates). Then, 1×10⁶ RBC-lysed BM nucleated cells were injected retroorbitally under isoflurane (Phoenix pharmaceuticals) anesthesia. Some mice that were depleted with DT and harvested on day 7 were treated intraperitoneally on days 1 and 4 after BMT with 200 mg/kg elemental iron (Ferrlecit, sodium ferric gluconate complex in sucrose, Sanofi Aventis). For reciprocal BMT studies, 1×10⁶ WT BM nucleated cells were infused into lethally irradiated WT (WT->WT) or DTR (WT->DTR) mice or 1×10⁶ DTR BM nucleated cells were infused into lethally irradiated WT (WT->DTR) or DTR (DTR->DTR) mice. For polycythemia vera experiments, 3.7×10⁶ RBC-lysed BM cells from C57BL/6 (WT) or JAK2V617F (JAK2) transgenic animals were infused into lethally irradiated C57BL/6-Ly5.2 mice. Mice were allowed to recover 5 weeks or 8 weeks prior to infusion of liposomes. In some experiments, mice were treated intraperitoneally daily with 100 mg/kg deferoxamine (Desferal, Novartis).

Quantitative real-time PCR (Q-PCR). 10,000 RBC-separated splenocytes were lysed in buffer from the Dynabeads RNA Microkit (Invitrogen) in accordance with manufacturer's instructions. Conventional reverse transcription, using the Sprint PowerScript reverse transcriptase (Clontech) was performed in accordance with the manufacturers' instructions. Q-PCR was performed with SYBR GREEN on an ABI PRISM 7900HT Sequence Detection System (Applied Biosystems). The PCR protocol consisted of one cycle at 95° C. (10 min) followed by 40 cycles of 95° C. (15 s) and 60° C. (1 min). Expression of glyceraldehyde-3-phosphate dehydrogenase (Gapdh) was used as a standard. The average threshold cycle number (CRtR) for each tested mRNA was used to quantify the relative expression of each gene: 2\hat{ }[Ct(Gapdh)-Ct(gene)]. Primers used are listed below: Bmp4 (fwd) ATTCCTGGTAACCGAATGCTG (SEQ ID NO:1), Bmp4 (rev) CCGGTCTCAGGTATCAAACTAGC (SEQ ID NO:2), Gapdh (fwd) TGTGTCCGTCGTGGATCTGA (SEQ ID NO:3), Gapdh (rev) CCTGCTTCACCACCTTCTTGA (SEQ ID NO:4).

5-fluorouracil challenge. Mice were injected with 5FU (250 mg/kg; Sigma) i.v. under isoflurane (Phoenix pharmaceuticals) anesthesia.

Sternum imaging. Sternal bones were fixed with 4% paraformaldehyde for 30 minutes, blocked with PBS containing 20% normal goat serum (NGS) for three hours, permeabilized with 0.1% Triton X-100+5% NGS overnight, permeabilized again with 0.3% Triton X-100 for 2 hours, and then stained with Ter119-PE for two nights. Three washes with PBS for 15 minutes/wash were used between each step. Slides were stained 1:1000 of 10 mg/ml Hoechst 33342 for 2 hours immediately prior to image acquisition. Images were acquired using a ZEISS AXIO examiner D1 microscope (Zeiss, Germany) with a confocal scanner unit, CSUX1CU (Yokogawa, Japan) and reconstructed in 3-D with Slide Book software (Intelligent Imaging Innovations).

Microarray. To purify mononuclear phagocyte populations for microarray, the gating strategy was modified from a previously published gating scheme (7). BM was sorted two times with a FACS Aria sorter (BD) to achieve >99% purity. Grlhi monocytes were identified by Gr-1+CD115+CD3-B220-. Grllo monocytes were identified by Gr-1-CD115+F4/80+CD3-B220-. Macrophages were identified as Gr1-CD115intF4/80+CD3-B220-SSClo. Microarray analysis of sorted cells was performed in collaboration with the Immunological Genome Project (Immgen).

VCAM1 blockade. Mice were infused i.v. with 10 mg/kg VCAM1 antibody (clone M/K 2.7) (Bio X Cell) or IgG from rat serum (Sigma) per infusion. For BMT experiments, mice were infused on days 1, 4, 7, 10, and 13 post-BMT.

Characterization of human bone marrow macrophages. Unprocessed fresh human BM aspirates were purchased from Lonza. Leukocytes were purified by harvesting the interface layer after Ficoll (GE Healthcare) separation. Populations were sorted using BSL2-level FACS Aria machine (BD) and cytospun as above. Photomicrographs were acquired using an upright Zeiss AxioPlan II at the MSSM Microscopy Shared Resource Facility.

Iron studies. Serum iron and UIBC were measured using an Iron/TIBC Reagent Set (Pointe Scientific) and transferrin saturation was calculated according to manufacturer's instructions.

Statistical analyses. Unless otherwise indicated in the figure legends, the unpaired Student's t test was used in all analyses. Data in bar graphs are represented as mean±SEM and statistical significance was expressed as follows: *, P<0.05; **, P<0.01; ***, P<0.001; n.s., not significant

### REFERENCES

1. Bessis, M. L'îlot érythroblastique, unite fonctionnelle de la moelle osseuse. Rev Hematol 13, 8-11 (1958).
2. Mohandas, N. & Prenant, M. Three-dimensional model of bone marrow. Blood 51, 633-643 (1978).
3. Lee, G., et al. Targeted gene deletion demonstrates that the cell adhesion molecule ICAM-4 is critical for erythroblastic island formation. Blood 108, 2064-2071 (2006).
4. Rhodes, M. M., Kopsombut, P., Bondurant, M. C., Price, J. O. & Koury, M. J. Adherence to macrophages in erythroblastic islands enhances erythroblast proliferation and increases erythrocyte production by a different mechanism than erythropoietin. Blood 111, 1700-1708 (2008).
5. Hanspal, M., Smockova, Y. & Uong, Q. Molecular identification and functional characterization of a novel protein that mediates the attachment of erythroblasts to macrophages. Blood 92, 2940-2950 (1998).
6. Chasis, J. A. & Mohandas, N. Erythroblastic islands: niches for erythropoiesis. Blood 112, 470-478 (2008).
7. Chow, A., et al. Bone marrow CD169+ macrophages promote the retention of hematopoietic stem and progenitor cells in the mesenchymal stem cell niche. The Journal of experimental medicine 208, 261-271 (2011).
8. Chow, A., Brown, B. D. & Merad, M. Studying the mononuclear phagocyte system in the molecular age. Nature reviews. Immunology 11, 788-798 (2011).
9. Miyake, Y., et al. Critical role of macrophages in the marginal zone in the suppression of immune responses to apoptotic cell-associated antigens. J Clin Invest 117, 2268-2278 (2007).
10. Crocker, P. R., Werb, Z., Gordon, S. & Bainton, D. F. Ultrastructural localization of a macrophage-restricted sialic acid binding hemagglutinin, SER, in macrophage-hematopoietic cell clusters. Blood 76, 1131-1138 (1990).
11. Manwani, D. & Bieker, J. J. The erythroblastic island. Current topics in developmental biology 82, 23-53 (2008).
12. Barbe, E., Huitinga, I., Dopp, E. A., Bauer, J. & Dijkstra, C. D. A novel bone marrow frozen section assay for studying hematopoietic interactions in situ: the role of stromal bone marrow macrophages in erythroblast binding. Journal of cell science 109 (Pt 12), 2937-2945 (1996).
13. Ramos, P., et al. Enhanced erythropoiesis in Hfe-KO mice indicates a role for Hfe in the modulation of erythroid iron homeostasis. Blood 117, 1379-1389 (2011).
14. Schroit, A. J., Madsen, J. W. & Tanaka, Y. In vivo recognition and clearance of red blood cells containing phosphatidylserine in their plasma membranes. The Journal of biological chemistry 260, 5131-5138 (1985).
15. Sadahira, Y., et al. Impaired splenic erythropoiesis in phlebotomized mice injected with CL2MDP-liposome: an experimental model for studying the role of stromal macrophages in erythropoiesis. J Leukoc Biol 68, 464-470 (2000).
16. Bessis, M. [Erythroblastic island, functional unity of bone marrow]. Rev Hematol 13, 8-11 (1958).
17. Cairo, G., Recalcati, S., Mantovani, A. & Locati, M. Iron trafficking and metabolism in macrophages: contribution to the polarized phenotype. Trends in immunology 32, 241-247 (2011).
18. Zhang, Z., et al. Ferroportinl deficiency in mouse macrophages impairs iron homeostasis and inflammatory responses. Blood 118, 1912-1922 (2011).
19. Harandi, O. F., Hedge, S., Wu, D. C., McKeone, D. & Paulson, R. F. Murine erythroid short-term radioprotection requires a BMP4-dependent, self-renewing population of stress erythroid progenitors. J Clin Invest 120, 4507-4519 (2010).
20. Paulson, R. F., Shi, L. & Wu, D. C. Stress erythropoiesis: new signals and new stress progenitor cells. Curr Opin Hematol 18, 139-145 (2011).
21. Hashimoto, D., et al. Pretransplant CSF-1 therapy expands recipient macrophages and ameliorates GVHD after allogeneic hematopoietic cell transplantation. The Journal of experimental medicine 208, 1069-1082 (2011).
22. Sadahira, Y., Mori, M. & Kimoto, T. Participation of radioresistant Forssman antigen-bearing macrophages in the formation of stromal elements of erythroid spleen colonies. Br J Haematol 71, 469-474 (1989).
23. Millot, S., et al. Erythropoietin stimulates spleen BMP4-dependent stress erythropoiesis and partially corrects anemia in a mouse model of generalized inflammation. Blood 116, 6072-6081 (2010).
24. Sadahira, Y., Yoshino, T. & Monobe, Y. Very late activation antigen 4-vascular cell adhesion molecule 1 interaction is involved in the formation of erythroblastic islands. The Journal of experimental medicine 181, 411-415 (1995).
25. Kohyama, M., et al. Role for Spi-C in the development of red pulp macrophages and splenic iron homeostasis. Nature 457, 318-321 (2009).
26. Gooi, H. C., et al. Marker of peripheral blood granulocytes and monocytes of man recognized by two monoclonal antibodies VEP8 and VEP9 involves the trisaccharide 3-fucosyl-N-acetyllactosamine. Eur J Immunol 13, 306-312 (1983).
27. Tippett, E., et al. Differential expression of CD163 on monocyte subsets in healthy and HIV-1 infected individuals. Plos One 6, e19968 (2011).
28. Xing, S., et al. Transgenic expression of JAK2V617F causes myeloproliferative disorders in mice. Blood 111, 5109-5117 (2008).
29. Tefferi, A., et al. Proposals and rationale for revision of the World Health Organization diagnostic criteria for polycythemia vera, essential thrombocythemia, and primary myelofibrosis: recommendations from an ad hoc international expert panel. Blood 110, 1092-1097 (2007).
30. Scott, L. M., Priestley, G. V. & Papayannopoulou, T. Deletion of alpha4 integrins from adult hematopoietic cells reveals roles in homeostasis, regeneration, and homing. Mol Cell Biol 23, 9349-9360 (2003).
31. Ulyanova, T., Jiang, Y., Padilla, S., Nakamoto, B. & Papayannopoulou, T. Combinatorial and distinct roles of alpha and alpha integrins in stress erythropoiesis in mice. Blood 117, 975-985 (2011).
32. Bungartz, G., et al. Adult murine hematopoiesis can proceed without beta1 and beta7 integrins. Blood 108, 1857-1864 (2006).
33. Thiele, J., et al. Macrophages and their subpopulations following allogenic bone marrow transplantation for chronic myeloid leukaemia. Virchows Archiv: an international journal of pathology 437, 160-166 (2000).
34. Thiele, J., et al. Erythropoietic reconstitution, macrophages and reticulin fibrosis in bone marrow specimens of CIVIL patients following allogeneic transplantation. Leukemia 14, 1378-1385 (2000).
35. Hartnell, A., et al. Characterization of human sialoadhesin, a sialic acid binding receptor expressed by resident and inflammatory macrophage populations. Blood 97, 288-296 (2001).
36. Lenox, L. E., Perry, J. M. & Paulson, R. F. BMP4 and Madh5 regulate the erythroid response to acute anemia. Blood 105, 2741-2748 (2005).
37. Coleman, D. L., Russell, E. S. & Levin, E. Y. Enzymatic studies of the hemopoietic defect in flexed mice. Genetics 61, 631-642 (1969).
38. Seggewiss, R. & Einsele, H. Hematopoietic growth factors including keratinocyte growth factor in allogeneic and autologous stem cell transplantation. Semin Hematol 44, 203-211 (2007).
39. Miller, C. B., et al. Impaired erythropoietin response to anemia after bone marrow transplantation. Blood 80, 2677-2682 (1992).
40. Heuser, M. & Ganser, A. Recombinant human erythropoietin in the treatment of nonrenal anemia. Ann Hematol 85, 69-78 (2006).
41. Zhan, H. & Spivak, J. L. The diagnosis and management of polycythemia vera, essential thrombocythemia, and primary myelofibrosis in the JAK2 V617F era. Clinical advances in hematology & oncology: H&O 7, 334-342 (2009).
42. Reddy, M. M., Deshpande, A. & Sattler, M. Targeting JAK2 in the therapy of myeloproliferative neoplasms. Expert opinion on therapeutic targets 16, 313-324 (2012).
43. Hume, D. A. & MacDonald, K. P. Therapeutic applications of macrophage colony-stimulating factor-1 (CSF-1) and antagonists of CSF-1 receptor (CSF-1R) signaling. Blood 119, 1810-1820 (2012).
44. Saito, M., et al. Diphtheria toxin receptor-mediated conditional and targeted cell ablation in transgenic mice. Nature biotechnology 19, 746-750 (2001).
45. Chen, K., et al. Resolving the distinct stages in erythroid differentiation based on dynamic changes in membrane protein expression during erythropoiesis. Proc Natl Acad Sci USA 106, 17413-17418 (2009).
46. Reeves, J. P., Reeves, P. A. & Chin, L. T. Survival surgery: removal of the spleen or thymus. Current protocols in immunology/edited by John E. Coligan . . . [et al.] Chapter 1, Unit 1 10 (2001).
47. Hoffmann-Fezer, G., et al. Biotin labeling as an alternative nonradioactive approach to determination of red cell survival. Annals of Hematology 67, 81-87 (1993).

1. A method of treating a subject having a blood disorder comprising erythropoietic stress, the method comprising administering to the subject an amount of an erythropoiesis-stimulating agent effective to treat the blood disorder.

2. The method of claim 1, wherein the erythropoiesis-stimulating agent comprises a plurality of CD169⁺ macrophages or a CSF-1 agonist.

3. The method of claim 2, wherein the erythropoiesis-stimulating agent comprises a plurality of CD169⁺ macrophages and wherein the CD169⁺ macrophages are allogeneic to, or syngeneic to, the subject.

4. The method of claim 1, wherein the blood disorder is selected from an anemia, from acute blood loss, acute or chronic hemolysis, a hemoglobinopathy, myeloablative injury, hematopoietic stem cell transplant, chemotherapy or irradiation-induced injury.

5-10. (canceled)

11. A method of determining the prognosis of an erythroid compartment in a subject having a condition comprising erythropoietic stress, comprising
obtaining a bone marrow sample and/or splenic sample from the subject and quantifying CD169⁺ macrophages in the sample(s),
comparing the amount of CD169⁺ macrophages quantified to a predefined reference amount, and
determining the prognosis of the erythroid compartment as a negative or a positive prognosis,
wherein an amount of CD169⁺ macrophages quantified in excess of the reference amount indicates a positive prognosis, and an amount of CD169⁺ macrophages quantified below the reference amount indicates a negative prognosis.

12. The method of claim 11, further comprising administering a blood product and/or a erythropoiesis-stimulating agent to a subject identified to be in need thereof by being identified as having negative prognosis by the method.

13. The method of claim 12, wherein the blood product is administered as a blood transfusion.

14. The method of claim 12, wherein the erythropoiesis-stimulating agent comprises a plurality of CD169⁺ macrophages or is a CSF-1 agonist.

15. The method of claim 14, wherein the erythropoiesis-stimulating agent comprises a plurality of CD169⁺ macrophages and the CD169⁺ macrophages are allogeneic to the subject or syngeneic to the subject.

16. The method of claim 11, wherein the condition is selected from an anemia, acute blood loss, acute or chronic hemolysis, a hemoglobinopathy, myeloablative injury, hematopoietic stem cell transplant, chemotherapy or irradiation-induced injury.

17. (canceled)

18-23. (canceled)

24. A composition comprising an erythropoiesis-stimulating agent for treating a subject having a blood disorder comprising erythropoietic stress.

25. The composition of claim 24, wherein the erythropoiesis-stimulating agent comprises a plurality of CD169⁺ macrophages or is a CSF-1 agonist.

26. The composition of claim 25, wherein the erythropoiesis-stimulating agent comprises a plurality of CD169⁺ macrophages and wherein the CD169⁺ macrophages are allogeneic to, or syngeneic to, the subject.

27. The erythropoiesis-stimulating agent of claim 25, wherein the blood disorder is selected from an anemia, acute blood loss, acute or chronic hemolysis, a hemoglobinopathy, myeloablative injury, hematopoietic stem cell transplant, chemotherapy or irradiation-induced injury.

28-33. (canceled)

34. The composition of claim 25, further comprising the plurality of CD169⁺ macrophages comprising CD169⁺ macrophages obtained from a biological sample, wherein the plurality of CD169⁺ macrophages is admixed with a pharmaceutically-acceptable carrier.

35. The composition of claim 34, wherein the concentration of CD169⁺ macrophages in the pharmaceutically-acceptable carrier is greater than the concentration of CD169⁺ macrophages in the same volume of the biological sample.

36. The composition of claim 25, wherein the plurality of CD169⁺ macrophages comprises isolated CD169⁺ macrophages, and further comprising a pharmaceutically-acceptable carrier.

37. The composition of claim 36, wherein the isolated CD169⁺ macrophages are enriched in the composition relative to the same volume in a human bone marrow sample or a human splenic sample.

38. The composition of claim 25, wherein the plurality of CD169⁺ macrophages are allogenic to or syngeneic to the subject.

39. The composition of claim 25, wherein the CSF-1 agonist is admixed with a pharmaceutically-acceptable carrier.
